(19) United States
(12) Patent Application Publication
(10) Pub. No.: US 2019/0351012 A1
(43) Pub. Date: Nov. 21, 2019

(54) TARGETING OF INTERFERON-STIMULATED GENE EXPRESSION TO TREAT INTERFERONOPATHIES

(71) Applicant: New York University, New York, NY (US)

(72) Inventors: Isabelle Marie, New York, NY (US); David Levy, New York, NY (US)

(21) Appl. No.: 16/417,755

(22) Filed: May 21, 2019

Related U.S. Application Data
(60) Provisional application No. 62/674,333, filed on May 21, 2018.

(51) Int. Cl. A61K 38/15; A61K 31/551; A61P 37/00

(52) U.S. Cl. CPC A61K 38/15; A61P 37/00; A61K 31/551

(57) ABSTRACT

Provided are methods of treating autoimmune diseases. The method entails administering to an individual in need of treatment one or more compositions that contain one or more inhibitors of histone deacetylatse (HDAC) and one or more inhibitors of BRD4. The method can include administering an inhibitor of binding of BRD4 to chromatin, an inhibitor of activation of BRD4 by phosphorylation, an inhibitor of signaling pathways leading to induction or increased expression of Type I interferon stimulated genes (ISG), and combinations thereof. The inhibitors may be administered at a sub-therapeutic dose, and may be administered concurrently or sequentially, in any combination.

### CROSS REFERENCE TO RELATED APPLICATIONS

This application claims priority to U.S. provisional patent application No. 62/674,333, filed May 21, 2018, the disclosure of which is incorporated herein by reference.

### STATEMENT REGARDING FEDERALLY SPONSORED RESEARCH

This invention was made with government support under grant number R01AI28900 awarded by the National Institutes of Health. The government has certain rights in the invention.

### BACKGROUND

Patients suffering from a number of autoimmune inflammatory syndromes, such a systemic lupus erythematosus (SLE) and rheumatoid arthritis (RA), exhibit elevated expression of type I interferon (IFN)-stimulated genes, and a variety of evidence suggests that these gene products contribute to disease pathogenesis. In particular, some rare monogenic forms of autoimmunity are caused by mutations in genes that directly impact the type IIFN pathway, and these diseases have become known as interferopopathies. Because of this, targeting the IFN pathway has become recognized as potential therapeutic approach. To date, therapies directed at neutralizing circulating type I IFN or its signaling receptor have shown efficacy in both animal models and human clinical trials. Significant limitations of this approach include an increased risk of infection and latent virus reactivation, due to the critical role the IFN pathway plays in antiviral responses, and an inability to readily titrate or modulate the therapeutic intervention for individual disease states. As such there is a continuing need for development of titrable therapeutic approaches for the treatment of autoimmune inflammatory diseases. The present disclosure is pertinent to this need.

### SUMMARY OF THE DISCLOSURE

In one aspect, this disclosure provides a method for treating an autoimmune disease by reducing the increased expression of Type I interferon stimulated genes (ISG). The expression can be titrated down so that the expression is reduced to normal or near-normal levels, but is not abrogated. This can result in alleviating one or more symptoms of the autoimmune disease without affecting the ability of an individual to mount acute response to infections. The present disclosure is based on the observations that IFN-stimulated gene transcription depends on HDAC activity, which is mediated, at least in part, by the BRD4 protein. BRD4 is an epigenetic reader and recognizes acetylated lysine residues on histones and other proteins and recruits transcription elongation factor to the transcription start site, while HDAC catalyzes the removal of acetyl groups on proteins. Inhibitors of BRD4 and HDAC have been used for cancer therapy. In the present disclosure, we surprisingly observed that by combining inhibitors of HDAC and BRD4 at lower than therapeutic amounts used in cancer treatments, a sufficient reduction in the expression of ISG can be obtained such that one of the symptoms of an autoimmune condition can be alleviated without significantly affecting the ability to fight infections. Such a combinatorial approach allows the use of lower doses for each inhibitor compared to the amount of each inhibitor if used by itself and can provide a viable approach to reducing inflammation without abrogating beneficial IFN signaling.

The method provided in the disclosure has a number of advantages, including the likelihood of employing small molecule inhibitors rather than biologies; targeting differential subsets of IFN action that might confer selective therapeutic effects; and modulating the degree of pathway inhibition with the goal of impeding chronic activation while preserving acute responses necessary for antiviral function. Potential targets that can have selectivity for impairing IFN signaling and function include unique aspects of ISG transcription.

In one aspect, this disclosure provides a method of treating an autoimmune disease comprising administering to an individual in need of treatment one or more inhibitors of HDAC and one or more inhibitors of BRD4. In embodiments, the method can include administering an inhibitor of binding of BRD4 to chromatin, an inhibitor of activation of BRD4 by phosphorylation, and/or an inhibitor of signaling pathways leading to induction or increased expression of Type I interferon stimulated genes (ISG), and combinations thereof. The inhibitors may be administered at a sub-therapeutic dose, and may be administered concurrently or sequentially, and in any combination.

The autoimmune disease may be characterized by an increased expression of ISG. The inhibitors of HDAC and BRD4 may be administered in the same composition or different compositions, at the same time or different times, by the same route or different routes, and over a same period of time or different periods of time. They may be administered sequentially or concurrently and the administrations may overlap. In one embodiment, one or both inhibitors are administered at a dosage which is lower than the dosage typically used for the inhibitor alone in the treatment of the indication. The lower dosage used for one or both inhibitors may be a sub-therapeutic dosage.

### BRIEF DESCRIPTION OF THE FIGURES

FIG. 1. Class I HDAC mediates ISG expression. (A, B) HeLa cells were treated with IFN-α for 6 h in the absence or presence of increasing concentrations of Romidepsin or TSA, as indicated. RNA was quantified by real-time RT-PCR for ISG54 and p21^{wAF1/Cip1} expression, normalized to GAPDH, and represented as fold induction over untreated cells. (C) HeLa cells were treated with IFN-α for 6 h in the absence or presence of increasing concentrations of romidepsin, RGFP966, or RGFP233, as indicated. RNA was quantified by real-time RT-PCR for IRF9 expression, normalized to GAPDH and represented as fold induction over untreated cells. HEK293 cells were transfected with siRNA against HDAC1, HDAC2, or E2F4 separately (D) or with a combination of HDAC1 and HDAC2 targeting oligonucleotides (E) and cells were stimulated with IFN-α for 6 h (α) in the presence (αT) or absence of TSA, as indicated. Whole cell extracts were analyzed for expression of the indicated proteins by western blotting (left panels). ISG54 expression was quantified by real-time RT-PCR, and represented as fold induction over untreated cells (right panels). (F) HEK293T cells were transfected with pcDNA3 (Ctl), HDAC1, or HDAC2 expression constructs, and ISG54 expression was quantified after stimulation with IFN-α for 10 h. Representative data from two experiments are shown. (G) Sin3A F/− and Sin3A F/− Sin3B F/− immortalized mouse embryonic fibroblasts expressing Cre-ERT2 were treated for 3 days with 40H-Tamoxifen (40H-T) or left untreated. Nuclear extracts were analyzed for expression of Sin3B by western blotting (middle panels). Expression of Sin3A (left panel) and IRF9 (right panel) mRNA were quantified by real-time RT-PCR and normalized to GAPDH mRNA expression. *Weak expression of Sin3B protein in Sin3A F/− Sin3B F/− fibroblasts prior to 40H-T treatment is likely due to some leakiness of Cre recombinase expression.

FIG. 2. HDAC inhibition does not prevent transcription in vitro or IFN stimulated chromatin remodeling in vivo. (A) Nuclear extracts from HEK293T cells expressing activated recombinant ISGF3 (STAT1, STAT2, IRF9 and JAK1) or transfected with vector (Ctl) were programmed with G6TI-CAT (G6TI) or p107 (ISG54) templates. Radiolabeled RNA transcripts were resolved by polyacrylamide-urea gel electrophoresis. Arrow and arrowhead indicate position of the transcripts from G6TI-CAT and p107, respectively. (B) HeLa cells were either left untreated or treated with IFN-α in the absence or presence of TSA (TSA in vivo) before nuclei were isolated for run-on transcription. Where indicated, TSA was added to the elongation reaction in vitro (TSA in vitro). Specific signals from γ-actin, ISG15, ISG56, 6-16, 9-27, IRF9 and GBP transcription were quantified following filter hybridization and normalized to the signal for GAPDH, arbitrarily set to 100. Representative data from three independent experiments are shown. FS2 human diploid fibroblasts were starved for 72 h before being treated with IFN-α for 7.5 h in the absence or presence of TSA. Mononucleosomal DNA fraction was purified from MNase-digested nuclei and analyzed for nucleosome positions by PCR quantitation of protected fragments. All samples were quantified by real-time PCR, except the—53—+1 ISG54 fragment, which was quantified by gel electrophoresis (C). The same mononucleosomal fractions were assayed for 2 fragments of the IFNβ promoter as control (D). Normalized protection factor for each sample was expressed proportionally to the signal obtained for fragment +40/+116 protected by the fixed nucleosome N2 at IFNβ promoter-proximal region, which was arbitrarily set at 100. Putative nucleosome positions are diagramed.

FIG. 3. RNAPII recruitment and activation do not require HDAC activity. (A) 2fTGH cells were either left untreated or treated with IFN-α in the absence or presence of TSA for the indicated time (min). ChIP assays were performed with antibodies against Pol II and recovered ISG56 promoter sequences were quantified by real-time PCR relative to input and represented as fold induction relative to untreated cells. (B) As in A, except that antibodies against phosphorylated Pol II at serine-5 in the CTD were used for immunoprecipitation, following 60 min IFN-α treatments. Recovered ISG54, ISG56 and ISG15 promoter sequences were quantified by real-time PCR relative to input and represented as fold induction relative to untreated cells. (C) As in A, except that cells were treated with IFN-α for 60 min and proximal, middle and distal fragments along the ISG54 gene (left panel) and ISG56 gene (right panel) were assayed. (D) As in A, except that antibodies against trimethylated lysine-4 on histone H3 were used. Promoter regions of ISG54, ISG56 and 6-16 genes were quantified by real-time PCR and reported as fold over the signal detected with a non-specific antibody.

FIG. 4. HDAC activity is required for P-TEFb recruitment. (A) HeLa cells were either left untreated or treated with IFN-α for 1 h in presence or absence of 100 or 200 nM of the CDK9 inhibitor flavopiridol (Flay). Using primers spanning intron-exon junctions, nuclear pre-mRNA abundance for ISG54 and RPS11 were quantified using real-time RT-PCR and normalized to GAPDH mRNA. (B) 2fTGH cells were either left untreated or treated with IFN-α in the absence or presence of TSA for 60 min. ChIP assays were performed with antibodies against CDK9 and recovered ISG56 and ISG15 promoter sequences were quantified by real-time PCR relative to input and represented as percent of the input signal. (C) BT474 cells were either left untreated or treated with IFN-α in the absence or presence of TSA after exposure to Geldanamycin (Gelda) for 24 h, as indicated. mRNA for ISG56, OAS2 and c-Myc were quantified by real-time RT-PCR and normalized to GAPDH. (D) Control and NELF-E KD cells were either left untreated or treated with IFN-α in the absence or presence of TSA. ISG56 mRNA was quantified using real-time RT-PCR and normalized to GAPDH mRNA abundance (left panel). Knockdown was verified by western blotting using anti-NELF-E antibodies (right panel). (E) Control and Spt5 KD cells were either left untreated or treated with IFNα in the absence or presence of TSA. ISG56, Spt5 mRNA, and ISG54 pre-mRNA were quantified using real-time RT-PCR and normalized to GAPDH mRNA abundance.

FIG. 5. BRD4 is required for ISG transcription. (A) HeLa cells were either left untreated or treated with IFN-α for 1 h in presence or absence of JQ-1 or HMBA. Using primers spanning intron-exon junctions, nuclear pre-mRNA for ISG54 and γ-actin were quantified by real-time RT-PCR and normalized to GAPDH mRNA. (B) Control and BRD4 KD cells were either left untreated or treated with IFN-α. ISG56 and BRD4 mRNA were quantified using real-time RT-PCR and normalized to GAPDH mRNA abundance. (C) HEK293T cells were transfected with pcDNA3 (Ctl) or Brd4 expression constructs along with a luciferase reporter driven by the ISG54 promoter. 24 h after transfection, cells were either left untreated or treated with IFN-α in the absence or presence of TSA before being assayed for luciferase activity (left panel). HEK293T cells were transfected with pcDNA3 (Ctl) or Brd4 expression constructs along with a luciferase reporter driven by the constitutive RSV promoter and assayed for luciferase activity after 36 h (right panel). (D) Hela cells were either left untreated or treated with IFN-α for 2 h in presence or absence of TSA or Anacardic acid (AA). Nuclear proteins were extracted as a soluble fraction and a chromatin bound fraction and analyzed by western blotting using anti-BRD4, H4-AcK5 and actin antibodies. (E) Hela cells were either left untreated or treated with IFN-α in the absence or presence of TSA or JQ-1 for 60 min. (F) ChIP assays were performed with antibodies against BRD4 and recovered ISG56, Mx1 and IRF9 TSS spanning sequences were quantified by real-time PCR, normalized to input, and represented as fold enrichment over the untreated sample.

FIG. 6. Combined HDAC and BRD4 inhibition is a potential therapy for type I interferonopathies. (A) HEK293T cells were either left untreated or treated with IFN-α for 6 h in presence or absence of JQ-1, TSA or a combination of the 2 inhibitors, as indicated. ISG56 and Viperin mRNA were quantified using real-time RT-PCR and normalized to GAPDH mRNA abundance. (B) hTert-immortalized human fibroblasts from a ISG15 deficient patient and a healthy donor (C21) were treated with IFN-α for 8 h, washed with PBS, and incubated in absence of IFN for 3 d. Where indicated, cells were treated with JQ-1, Romidepsin or a combination of the 2 drugs for the final 24 h prior RNA extraction. Mx1 and Viperin mRNA were quantified using real-time RT-PCR and normalized to GAPDH mRNA abundance. (C) As in (B), except that hTert-immortalized fibroblasts from a USP18 deficient patient were used. (D) As in (B), except that nuclear pre-mRNA for ISG54 from ISG15-deficient cells was scored. (E) As in (D), except that pre-mRNA for ISG54 from hTert-immortalized USP18 deficient fibroblasts was quantified.

### DESCRIPTION OF THE DISCLOSURE

We have characterized unique aspects of IFN-stimulated transcription that can be targeted pharmacologically. Specifically, we have found that the transcription elongation factor BRD4 is required for IFN-stimulated gene expression and that its availability for mediating transcription can be modulated by inhibiting histone deacetylases (HDAC), which cause an increase in histone acetylation and therefore trap BRD4 in a manner that is unavailable to mediate IFN-stimulated transcription. Combinations of direct BRD4 inhibitors with HDAC inhibitors were capable of resolving the autoinflammatory gene expression pattern of mutant cells from interferonopathy patients, demonstrating the efficacy of this approach.

The present disclosure of combined pharmacologic targeting of multiple steps unique to IFN-stimulated transcription as treatment for IFN-dependent autoinflammatory syndromes can have a number of advantages over current therapies, including the likelihood of employing small molecule inhibitors rather than biologics; targeting differential subsets of IFN action that might confer selective pathogenic effects; and modulating the degree of pathway inhibition with the goal of impeding chronic activation while preserving acute responses necessary for antiviral function.

The term “treatment” as used herein refers to reduction in one or more symptoms or features associated with the presence of the particular condition being treated. Treatment does not necessarily mean complete remission, nor does it preclude recurrence or relapses. For example, treatment in the present disclosure means reducing or inhibiting one or more symptoms associated with IFN-dependent autoinflammatory syndromes.

The term “therapeutically effective amount” as used herein in reference to a single agent is the amount sufficient to achieve, in a single or multiple doses, the intended purpose of treatment. The term “therapeutically effective amounts” of multiple agents if it is a combination refers to the amount(s) of each agent in the combination sufficient for the combination to achieve, in a single or multiple doses, in the same or separate compositions the intended purpose of treatment. In particular, this disclosure provides the administration of therapeutically effective amounts of agents as a combination (whether as a single composition or separate compositions) that while alleviating one or more symptoms of an IFN-dependent autoinflammatory syndrome, still preserves the ability to mount acute responses necessary for antiviral function. The term “sub-therapeutic amount” as used herein means an amount of an agent that by itself is not considered suitable for treatment of a condition. However, as described herein when a sub-therapeutic amount of an inhibitor (e.g., HDAC inhibitor) is combined with a therapeutic or sub-therapeutic amount of another inhibitor (e.g., BRD4 inhibitor), a titration of their effects on ISG expression may be realized. Similarly, when a sub-therapeutic amount of an inhibitor (e.g., BRD4 inhibitor) is combined with a therapeutic or sub-therapeutic amount of another inhibitor (e.g., HDAC inhibitor), a titration of their effects on ISG expression may be realized. An example of a therapeutic dose for Romidepsin is 14 mg/m², and a dose for BRD4i is 250 nM.

For example, an effective amount to treat an IFN-dependent autoinflammatory syndrome is an amount sufficient to alleviate symptoms of the autoinflammatory syndrome. For example, symptoms associated with an autoinflammatory syndrome that can result from increased ISG expression can be arthralgia, fever, arthritis, fatigue, skin rash, dry eyes, headaches, and other inflammatory symptoms. The exact amount desired or required will vary depending on the particular compound or composition used, its mode of administration, patient specifics and the like. Appropriate effective amounts can be determined by one of ordinary skill in the art informed by the present disclosure.

Where a range of values is provided in this disclosure, it should be understood that each intervening value, to the tenth of the unit of the lower limit between the upper and lower limit of that range, and any other intervening value in that stated range is encompassed within the invention, unless clearly indicated otherwise. The upper and lower limits of these smaller ranges may independently be included in the smaller ranges encompassed within the disclosure.

In one aspect, this disclosure provides a method for treating an autoimmune disease comprising controlled inhibition of the expression of interferon stimulated genes such that while at least one or more symptoms of the autoimmune disease are alleviated, the ability of an individual to mount acute response to infections is not compromised. In one embodiment, the method comprises administering to an individual who is afflicted with an autoimmune disease one or more inhibitors of HDAC and one or more inhibitors of BRD4. It should be recognized that inhibitors of HDAC may also inhibit BRD4 or vice versa. In embodiments, the individual to which a combination of agents as described herein has been diagnosed with or is suspected of having an autoimmune disease. The autoimmune disease may be associated with an increased expression of ISG. In embodiments, the individual has not been diagnosed with cancer. In embodiments, the individual has not been previously treated with an HDAC or BRD4 inhibitor, prior to an initial treatment as described herein. In embodiments, the individual has been diagnosed with any of systemic lupus erythematosus (SLE), rheumatoid arthritis (R A, Sjögren syndrome, Aicardi-Goutiéres syndrome, STING-associated vasculopathy with onset in infancy, Singleton-Merten syndrome, Spastic paraparesis, Pseudo-TORCH syndrome, Type I diabetes, or Radiation-resistant cancers.

HDAC inhibitors are known in the art. For example, Romidepsin ((1S,4S,7Z,10S,16E,21R)-7-ethylidene-4,21-diisopropyl-2-oxa-12,13-dithia-5,8,20,23-tetrazabicyclo[8.7.6]tricos-16-ene-3,6,9,19,22-pentone) is a known HDAC inhibitor. Other known inhibitors of HDAC include Belinostat ((2E)-N-Hydroxy-3-[3-(phenylsulfamoyl)phenyl]prop-2-enamide), CUDC-101 (7-((4-((3-ethynylphenyl)amino)-7-methoxyquinazolin-6-yl)oxy)-N-hydroxyheptanamide), Dacinostat ((E)-N-hydroxy-3-[4-[[2-hydroxyethyl-[2-(1H-indol-3-yl)ethyl]amino]methyl]phenyl]prop-2-enamide) and Panobinostat ((2E)-N-hydroxy-3-[4-({[2-(2-methyl-1H-indol-3-yl)ethyl]amino}methyl)phenyl]acrylamide). These are available commercially, such as from Adooq Bioscience. Some HDAC inhibitors are described in U.S. Patent application publication no. 20180086750, 20170349540, and 20170305900, all incorporated herein by reference. Another example is RGFP233.

BRD4 inhibitors are also known in the art. For example, ARV-825 (2-((S)-4-(4-chlorophenyl)-2,3,9-trimethyl-6H-thieno[3,2-f][1,2,4]triazolo[4,3-a][1,4]diazepin-6-yl)-N-(4-(2-(2-(2-(2-((2-(2,6-dioxopiperidin-3-yl)-1,3-dioxoisoindolin-4-yl)amino)ethoxy)ethoxy)ethoxy)ethoxy)phenyl)acetamide), JQ-1 ((6S)-4-(4-Chlorophenyl)-2,3,9-trimethyl-6H-thieno[3,2-f][1,2,4]triazolo[4,3-a][1,4]diazepine-6-acetic acid 1,1-dimethylethyl ester), Silmitasertib (5-[(3-Chlorophenyl)amino]benzo[c]-2,6-naphthyridine-8-carboxylic acid). Other BRD4 inhibitors are disclosed in, for example, U.S. Patent application publication no. 20170304315, and 20170226065, all incorporated herein by reference. In non-limiting embodiments, ARV-825 targets BRD4 for proteolytic degradation, JQ-1 inhibits BRD4 binding to chromatin, and Silmitasertib inhibits BRD4 phosphorylation. Other inhibitors that can be used include signaling inhibitors (such as inhibitors of JAK signaling) including BMS-986165 (Bristol-Myers Squibb), Ruxolitinib ((3R)-3-Cyclopentyl-3-[4-(7H-pyrrolo[2,3-d]pyrimidin-4-yl)pyrazol-1-yl]propanenitrile), Cerdulatinib (4-(cyclopropylamino)-2-((4-(4-(ethylsulfonyl)piperazin-1-yl)phenyl)amino)pyrimidine-5-carboxamide), and Baricitinib (2-(3-(4-(7H-pyrrolo[2,3-d]pyrimidin-4-yl)-1H-pyrazol-1-yl)-1-(ethylsulfonyl)azetidin-3-yl)acetonitrile). In one embodiment, these inhibitors are used in addition to the HDAC or BRD4 inhibitors. All samples described herein displayed IFN responsiveness, with enhanced responses correlating with disease status.

Generally, the therapeutic dose of inhibitors is in the ˜1 μM range. In one embodiment, the inhibitors are used in a sub-therapeutic range. For example, inhibitors may be used at less than 1 μM range. In one example, the HDAC and the BRD4 inhibitors can be used at from 1 to 500 nM and all values therebetween. In one embodiment, the HDAC and the BRD4 inhibitors can be used at from 1 nm to 1 μM and all values therebetween. In embodiments, inhibitors are administered to patients in sufficient quantities to achieve peak plasma concentrations in the therapeutic or subtherapeutic target ranges between 1 nM and 1 μM. These or other plasma concentrations can for an individual agent or patient can be determined by pharmacodynamics studies that will be apparent to those skilled in the art, given the benefit of the present disclosure. This analysis will define which cell types in an individual patient displays enhanced IFN responsiveness, which can subsequently be used as a biomarker for assessing efficacy of treatment regimens with the composition and methods described herein.

Administration of the present compositions, separately or as a single composition, as described herein can be carried out using any suitable route of administration known in the art. For example, the compositions may be administered via intravenous, intramuscular, intraperitoneal, intracerobrospinal, subcutaneous, intra-articular, intrasynovial, oral, topical, or inhalation routes. The compositions may be administered parenterally or enterically. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated.

The inhibitors of the present disclosure can be provided in pharmaceutical compositions for administration by combining them with any suitable pharmaceutically acceptable carriers, excipients and/or stabilizers. Examples of pharmaceutically acceptable carriers, excipients and stabilizer can be found in Remington: The Science and Practice of Pharmacy (2005) 21st Edition, Philadelphia, Pa. Lippincott Williams & Wilkins. For example, suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG). The pharmaceutical compositions may comprise other therapeutic agents.

Prior to, during, or after termination of treatment for a particular indication, the effect of the administrations on IFN stimulated gene expression can be evaluated. In general there are known to be about 200 IFN stimulated genes. All of the known genes or a subset of genes can be evaluated for proper titration of the dosage or to confirm the administration is not having an adverse effect on the ability to mount response to infections. Ability to mount a response to infection can be measured ex vivo by using blood samples obtained from patients under treatment. The level of expression of one or more the IFN stimulated genes may be compared to a reference value obtained prior to initiation of the treatment or a reference value that represents a normal level of expression in the absence of the autoimmune disease.

The following examples are provided as illustrative examples and are not intended to be restrictive in any way.

### Example 1

ISG Transcription Requires Class I HDAC Activity.

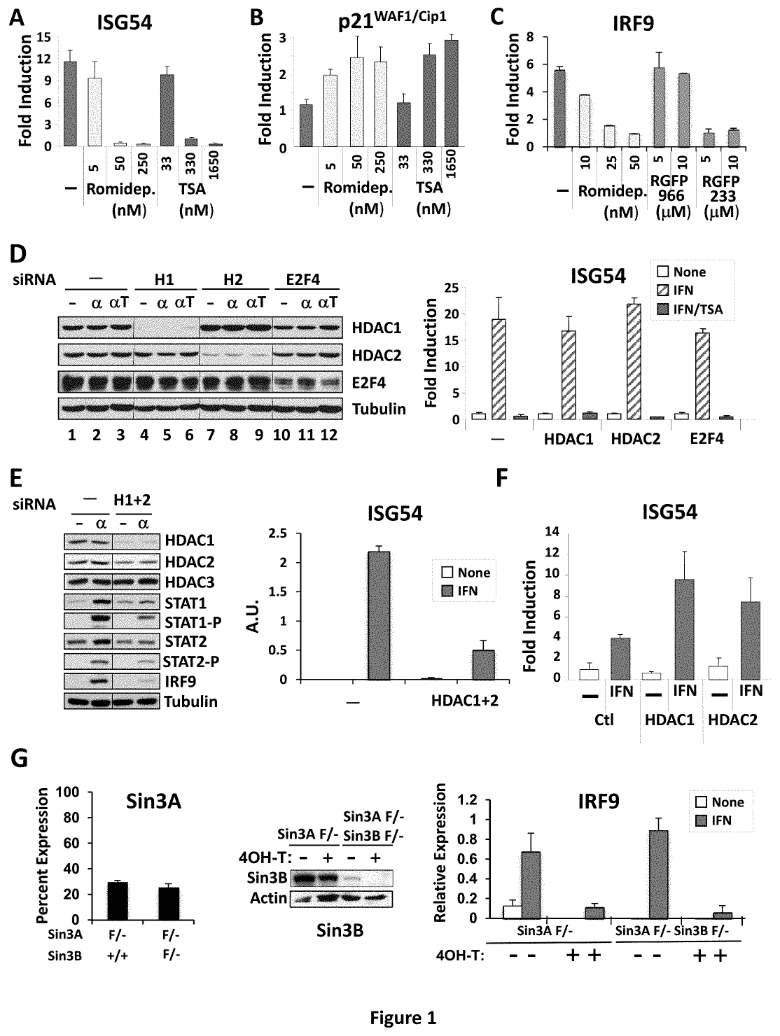

Trichostatin A (TSA) is a potent general inhibitor of class I and II HDAC enzymes, while valproate (VPA), which also inhibits IFN-stimulated gene expression, preferentially inhibits class I enzymes. We tested the efficacy of romidepsin, an inhibitor with high specificity for class I HDAC. Cells were stimulated with IFN in the presence of increasing concentrations of romidepsin ranging from 5-250 nM, since its IC50 for class I HDAC is approximately 40 nM and for class II is greater than 500 nM (Furumai et al., 2002, Cancer Res. 62:4916-4921). Romidepsin inhibited ISG54 expression partially at 5 nM and completely at 50 and 250 nM, equivalent to the effect of inhibitory concentrations of TSA (FIG. 1A). As control, both romidepsin and TSA treatments led to p21^{Cip1/wAF1} expression (FIG. 1B), indicative of HDAC inhibition. We extended this observation to another ISG, IRF9, showing that romidepsin inhibits IRF9 expression in a dose dependent manner with complete inhibition around 50 nM. In order to further identify what HDAC enzyme is involved in ISG transcription, we tested the effect of RGFP966, an inhibitor with selective specificity for HDAC3, and RGPF233, which preferentially inhibits HDAC1 and 2 (Park et al., 2004, Clin. Cancer Res. 10:5271-5281). Interestingly, RGFP966 had very little effect on ISG transcription, while RGFP233 almost completely abrogated ISG expression (FIG. 1C).

Pharmacologic inhibition by romidepsin and RGFP233 indicates HDAC1 and 2 are required for ISG transcription. We therefore depleted HDAC 1 and 2 by RNA interference. Knockdown of HDAC1 or HDAC2 individually did not affect IFN-stimulated gene induction (FIG. 1D, right panel), in spite of significantly reduced levels of the respective HDAC proteins (left panel). Interestingly, knockdown of HDAC2 led to a commensurate increase in HDAC1 levels (FIG. 1D, lanes 7-9). These two proteins exist together in several multiprotein deacetylase complexes, suggesting likely functional redundancy between them. Therefore, we simultaneously depleted HDAC1 and HDAC2. Reduction of HDAC1 and HDAC2 together led to a significant impairment in ISG54 induction in response to IFN (FIG. 1E, right panel), in spite of only partial knockdown of the HDAC2 protein (FIG. 1E, left panel), suggesting that these two enzymes together are critical for IFN responsiveness. As expected, depletion of the control protein E2F4 had no effect on ISG54 induction (FIG. 1D). We also examined expression of the IFN-inducible protein IRF9, as another measure of IFN responsiveness. IRF9 was highly induced in IFN-stimulated cells. However, knockdown of HDAC1 and 2 led to a significant impairment of IRF9 induction (FIG. 1E). STAT1 and to a lesser extent STAT2 are also IFN-inducible proteins. Their induction was also impaired by HDAC knockdown (FIG. 1E), although the ability of residual protein to be phosphorylated was largely unimpaired.

We ectopically expressed HDAC1 and HDAC2 by transfection in 293T cells, which led to a sensitized response to IFN (FIG. 1F). Heightened expression of HDAC2 caused a 50% increase in IFN-stimulated ISG54 expression, while HDAC1 caused a greater than 100% increase (FIG. 1F), an effect that was not observed following expression of a catalytically-impaired HDAC1 mutant or of other deacetylases (data not shown). Expression of neither enzyme affected basal expression of ISG54, which remained fully dependent on IFN stimulation.

HDAC1 and 2 are members of Sin3 complexes, which in mammalian cells contain either Sin3A or Sin3B and are commonly associated with gene repression. We further investigated the requirement for Sin3A and B proteins. Deletion of Sin3A is known to be incompatible with cell growth (Dannenberg et al., 2005, Genes Dev. 19:1581-1595). Therefore, we examined IFN responses in cells containing a conditional allele of Sin3A following acute deletion, in the presence or absence of the related Sin3B gene. Immortalized mouse fibroblasts containing a single conditional allele of Sin3A (Sin3A-F/−) and expressing a tamoxifen-regulated Cre recombinase protein, with or without deletion of Sin3B (Sin3B+/+ or F/−), were examined for ISG expression before and after treatment with 4-hydroxy-tamoxifen and/or IFN (FIG. 1G). Tamoxifen treatment significantly reduced the expression of Sin3A mRNA (left panel), whether or not Sin3B was expressed (middle panel). Loss of Sin3A significantly impaired induction of mouse IRF9 in response to IFN treatment (FIG. 1G, right panel), while loss of Sin3B had no effect on gene induction. Interestingly, loss of either Sin3A or Sin3B lowered basal expression of IRF9, indicating a possible role for IFN priming in maintaining basal expression levels. Residual IFN responsiveness was detected in Sin3-mutant cells, possibly due to incomplete loss of Sin3 complexes or to redundant activity from non-Sin3-containing HDAC complexes.

### Example 2

HDAC Inhibition does not Prevent Transcription In Vitro.

To determine if HDAC activity was required for transcriptional initiation, we adopted an in vitro transcription system using a genomic segment containing the promoter of the ISG54 gene as template. In vitro transcription reactions were programmed with nuclear extracts from 293T cells with or without ISGF3.

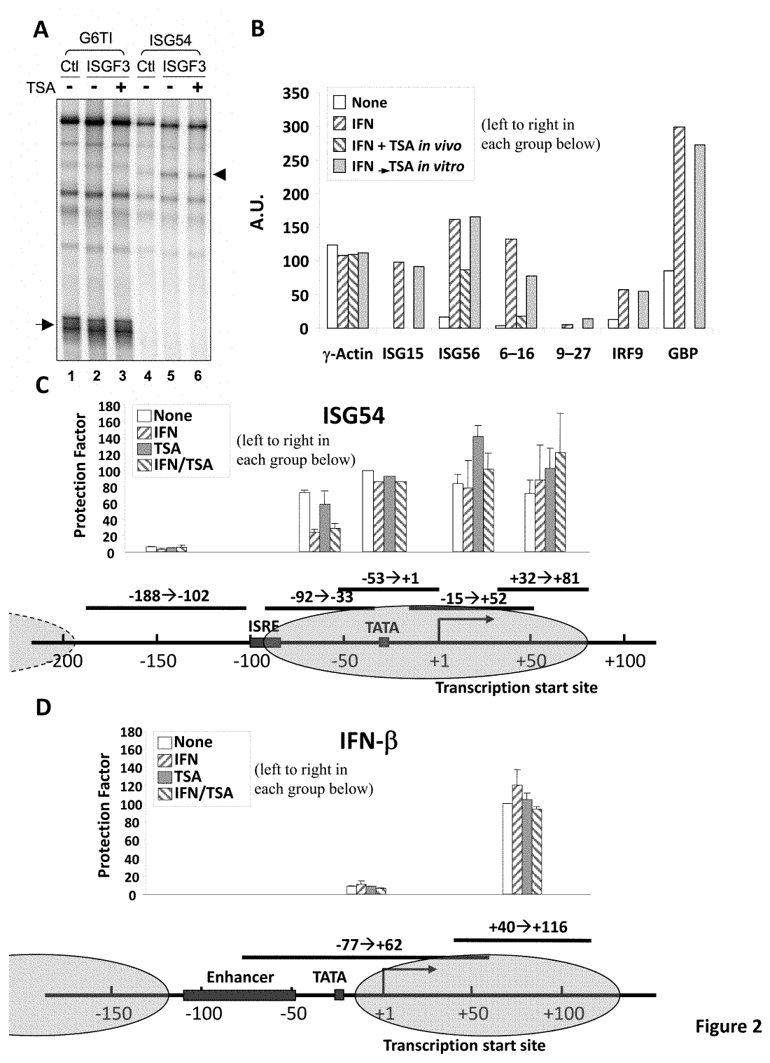

293T cell extracts supported basal transcription from a control template (G6TI) driven by Sp1, as indicated by a correctly initiated run-off product (FIG. 2A, lanes 1-3, arrow). However, transcription from the ISG54 promoter was only supported by nuclear extracts containing ISGF3 (FIG. 2A, lanes 5-6, arrowhead). The presence of activated ISGF3 resulted in a correctly initiated transcript from the ISG54 template without affecting transcription from the control, reflecting the known specificity of this transcription factor. Significantly, neither basal nor ISGF3-dependent transcription was inhibited by inclusion of TSA in the reaction, indicating that HDAC activity was not required for transcription in vitro on naked DNA templates (FIG. 2A, lanes 3 and 6).

Run-off transcription is largely a measure of transcriptional initiation. We asked whether HDAC activity was required for transcription in isolated nuclei, a measure of in vitro elongation of polymerase molecules previously initiated in vivo. Nuclei were isolated from cells untreated or treated with IFNα, pulse-labeled in vitro with radioactive nucleotides, and specific transcriptional elongation was quantified by filter hybridization (FIG. 2B). Transcription of several example ISGs was stimulated in nuclei isolated from IFN treated cells, while housekeeping gene transcription was equivalent regardless of how the cells were treated prior to nuclei isolation. Nuclei from cells stimulated with IFN in the presence of TSA displayed impaired ISG transcription, consistent with a requirement for HDAC activity for IFN-stimulated gene expression in vivo. However, similar to the in vitro run-off result, addition of TSA to the in vitro nuclear run-on reaction failed to affect either housekeeping or ISG transcription. Therefore, HDAC activity does not appear to be required for polymerase initiation in vitro or for polymerase elongation in vitro, at least for polymerases previously initiated and committed for elongation in vivo. We note that this assay, which produces relatively short bursts of transcriptional elongation, may not be sensitive to regulators of transcriptional processivity.

### Example 3

IFN-Stimulated Chromatin Remodeling.

The involvement of HDAC and Sin3-containing complexes in ISG expression prompted us to examine IFN-dependent changes in chromatin architecture. To this end, we explored the nucleosome environment of the ISG54 gene by micrococcal nuclease (MNase) protection. The mononucleosomal genomic DNA fraction was recovered from untreated or IFN-stimulated human diploid fibroblasts following MNase digestion of nuclei, and segments of the ISG54 promoter were quantified in the recovered DNA by using PCR primers tiled across the ISG54 promoter-proximal region. Efficient recovery of a specific DNA segment is indicative of protection from enzyme digestion by a placed nucleosome, while relative sensitivity to digestion is indicative of nucleosome-free or randomly placed nucleosome regions. As control, we examined the MNase sensitivity of the IFNβ promoter proximal region, documenting the nucleosome-free and fixed nucleosome elements that were unaffected by IFN treatment.

The ISG54 promoter distal region upstream of the ISRE was relatively nucleosome free and was not affected by IFN or TSA treatments (FIG. 2C). In contrast, genomic segments surrounding the TATA box and the transcriptional initiation site were relatively MNase resistant (FIG. 2C), equivalent to the nuclease resistance of control loci, such as the IFNβ promoter proximal region (FIG. 2D). Again, nuclease sensitivity was minimally affected by IFN or TSA treatments. The only ISG54 promoter region affected by IFN treatment was the −92 to −33 segment containing the ISRE. This region was resistant to digestion in unstimulated cells but became substantially more sensitive following IFN treatment (FIG. 2C), indicative of an alteration of nucleosome positioning. However, increased nuclease sensitivity following IFN treatment was not prevented by co-treatment with TSA.

These results indicate that the ISG54 ISRE and promoter-proximal region are packaged in a nucleosome, bounded by a relatively nucleosome-free region flanking the ISRE, which is altered in response to IFN.

### Example 4

RNAPII Recruitment and Activation do not Require HDAC Activity.

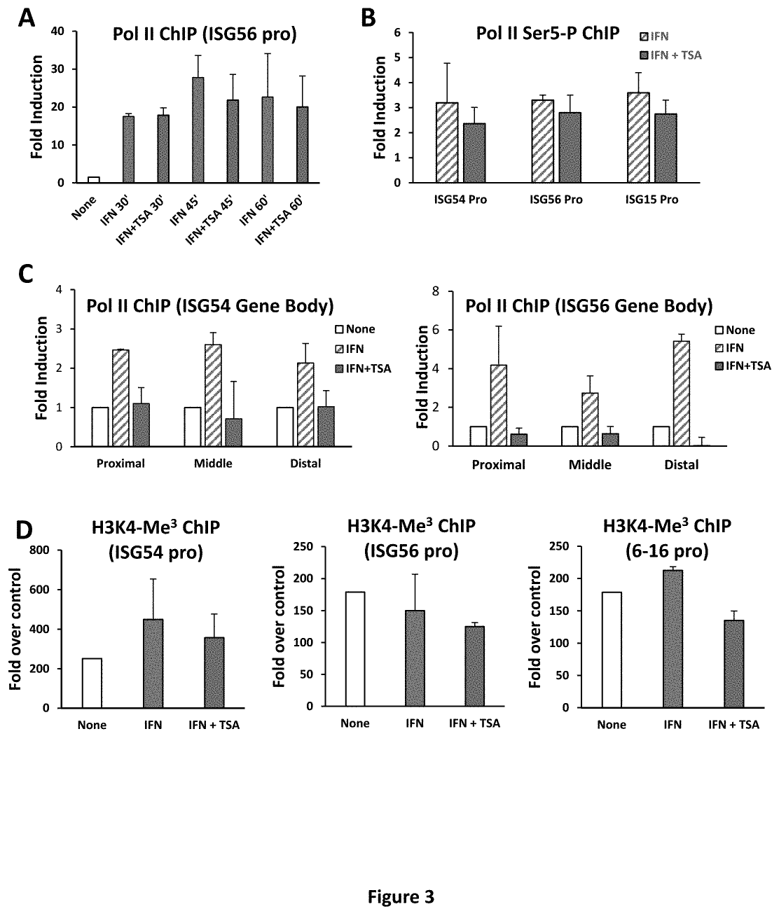

Since RNAPII recruitment to chromatin is a prerequisite for transcription, we asked whether this step required HDAC activity. ChIP analysis of RNAPII on the ISG56 promoter revealed recruitment in response to IFN stimulation that was rapid, robust, and time-dependent (FIG. 3A). RNAPII was readily detectable on the promoter after 30 min of IFN treatment, and continued to increase in abundance at 45 and 60 min post-treatment. Strikingly, promoter recruitment of RNAPII was largely unaffected by blocking HDAC activity at 30 min. post-treatment, and was only marginally reduced after 45 min and 1 h (FIG. 3A). The presence of RNAPII at ISG promoters even when transcription was blocked by HDAC inhibition suggests that HDAC inhibition results in a poised promoter configuration. One of the hallmarks of transcriptional initiation is RNAPII activation by carboxyl-terminal phosphorylation, with Ser 5 phosphorylation by TFIIH considered indicative of promoter clearance following preinitiation complex formation. RNAPII recruited to ISG promoters in response to IFN treatment was phosphorylated on Ser 5 regardless of HDAC inhibition (FIG. 3B). RNAPII was also recruited to the body of transcription units in response to IFN (FIG. 3C), although the density of RNAPII within the gene body was lower than that at the promoter. Significantly, RNAPII recruitment within the ISG56 and ISG54 transcription units was largely abrogated in the absence of HDAC activity (FIG. 3C), consistent with the observed inhibition of gene expression. These results are consistent with impairment of ISG transcription following HDAC inhibition, with RNAPII recruitment within gene bodies indicative of HDAC-dependent polymerase transit during transcription but promoter recruitment occurring even in absence of transcription.

A common hallmark of active or potentially active genes is the presence of trimethylation on lysine 4 of histone H3 (H3K4me3) at promoter or transcriptional start regions. This modification usually occurs subsequent to the assembly of the general transcription machinery on the promoter. Unexpectedly, we detected high levels of H3K4me3 on ISG promoters (FIG. 3D), even prior to IFN stimulation, hundred fold above control. However, neither IFN stimulation nor TSA co-treatment significantly affected the level of this histone mark. This suggests that ISG promoters exist in a poised state, ready for RNAPII recruitment and transcriptional activation. Taken together, these results demonstrate that while RNAPII can be efficiently recruited by STAT2, incorporated into a pre-initiation complex at ISG promoters, and serve as a substrate for modification by TFIIH, it fails to successfully transit the transcription unit in the absence of HDAC activity, resulting in aborted transcription. These epigenetic patterns strongly suggest that IFN stimulation and ISGF3 recruitment lead to a poised promoter architecture in which RNAPII is recruited and phosphorylated on Ser 5; however, committed elongation is dependent on HDAC activity.

### Example 5

HDAC Activity is Required for P-TEFb Recruitment.

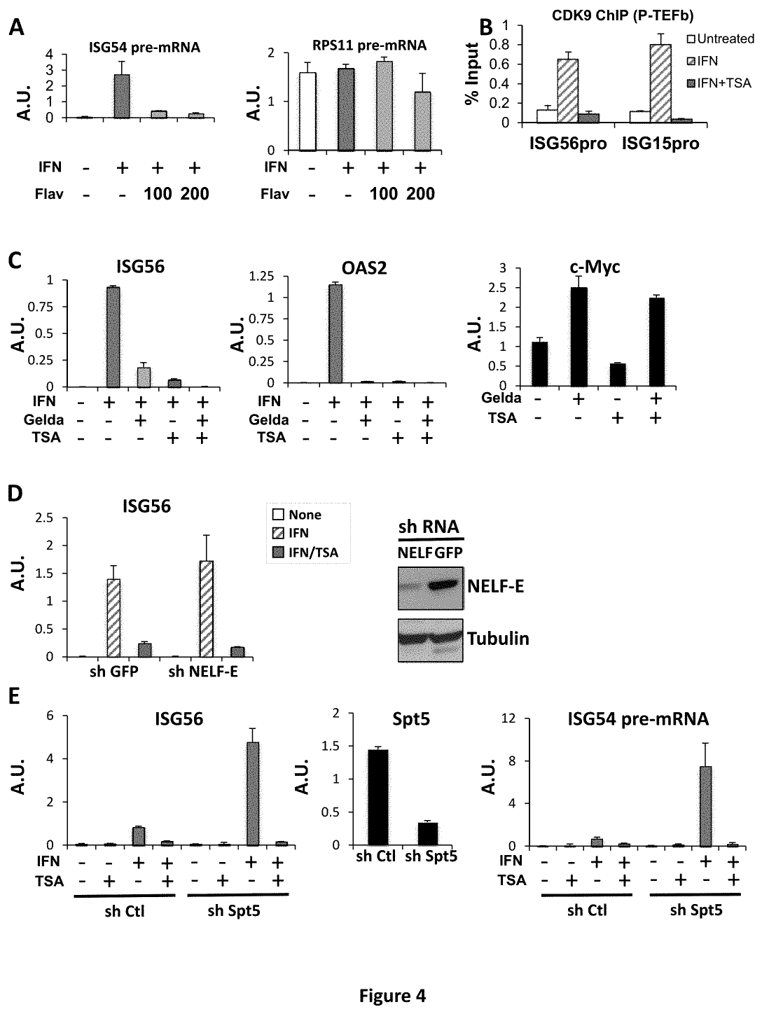

A crucial step driving the transition from promoter clearance to productive elongation is the recruitment of the CDK9/cyclin T1-containing P-TEFb complex. To test the importance of P-TEFb for ISG transcription, we used flavopiridol, a CDK9-selective inhibitor. To examine potential effects on transcription, we scored the abundance of unspliced pre-mRNA transcripts in the nucleus. Flavopiridol inhibited ISG54 induction at concentrations that did not prevent transcription of constitutively expressed RPS11 (FIG. 4A, right panel). Higher concentrations of flavopiridol inhibited expression of all genes tested (data not shown). These results suggest that ISG transcription is acutely sensitive to the action of P-TEFb relative to the sensitivity of constitutively expressed genes.

To directly probe the involvement of P-TEFb in ISG expression, we assessed its recruitment to chromatin in response to IFN treatment. P-TEFb was absent from ISG promoters prior to IFN treatment (FIG. 4B), but was rapidly recruited to the promoters of both ISG56 and ISG15, as measured by ChIP for CDK9. Notably, chromatin-bound CDK9 was not observed in cells treated with IFN in the presence of TSA, suggesting that P-TEFb recruitment required HDAC activity and correlated with the appearance of Ser 2 phosphorylated RNAPII and the transition to elongating transcription.

In addition to the RNAPII carboxyl-terminal tail, another CDK9 substrate is the negative elongation factor, NELF. It has been recently suggested that transcriptional inhibition by HDAC inhibitors in a different biological context was dependent on HSP90 function, whose ability to stabilize the NELF complex and therefore block elongation was dependent on HDAC activity (Greer et al., 2015, Cell Rep 13:1444-1455). Greer et al. showed that repression of ERBB2 and MYC expression by HDAC inhibitors is antagonized by geldanamycin treatment, a potent HSP90 inhibitor that led to loss of NELF and rescue of elongation. However, geldanamycin destabilization of NELF failed to rescue ISG transcription from HDAC inhibition; instead, ISG expression was partially inhibited by geldanamycin alone. However, repressed c-Myc expression was normalized by concomitant TSA and geldanamycin treatment (FIG. 4C).

We also tested more directly whether NELF dismissal could circumvent the HDAC requirement for ISG transcription. The NELF complex was depleted by RNA interference targeting NELF-E, since depletion of any of the 4 NELF subunits leads to functional loss of the entire complex. Again, destabilization of the NELF complex through reduced NELF-E did not significantly alter ISG induction in response to IFN and failed to rescue gene expression in absence of HDAC activity (FIG. 4D), suggesting that either the NELF complex does not play a prominent role in ISG regulation or that redundancy with other complexes precludes any remarkable effect.

The transcription elongation factor DRB sensitivity-inducing factor (DSIF) is another target of CDK9 that has been implicated in promoter proximal pausing of Pol II. To test its role, we targeted Spt5, the major subunit of DSIF, by RNA interference. Interestingly, downregulation of Spt5 substantially increased ISG induction in response to IFN, but, similar to NELF depletion, failed to rescue gene expression when HDAC activity was inhibited (FIG. 4E, left panel). DSIF also plays a role in coordinating elongation with mRNA splicing and nuclear export, which could complicate interpretation of changes in mRNA abundance in its absence. To ascertain whether the role of DSIF in ISG regulation was transcriptional, we scored the abundance of ISG54 pre-mRNA transcripts as a measure of ongoing transcription after IFN stimulation. The effect of DSIF depletion was even greater on primary transcripts, suggesting that DSIF plays a major role as a negative regulator of ISG transcription, possibly as a pause-release factor (FIG. 4E, right panel). However, ISG transcription remained sensitive to HDAC inhibition even in absence of the negative regulation imposed by DSIF. These results demonstrate that displacement of NELF or inactivation of DSIF are insufficient, at least individually, to allow transition from transcription initiation to transcriptional elongation in absence of HDAC activity, suggesting that P-TEFb likely targets additional substrates to enhance elongation. Taken together, these results are consistent with a model in which HDAC activity following IFN stimulation is required for P-TEFb recruitment to ISG promoters in order to relieve promoter proximal pausing and enhance elongation, but that inactivation of previously identified negative regulators is not sufficient to explain the HDAC requirement.

### Example 6

BRD4 is Required for ISG Transcription.

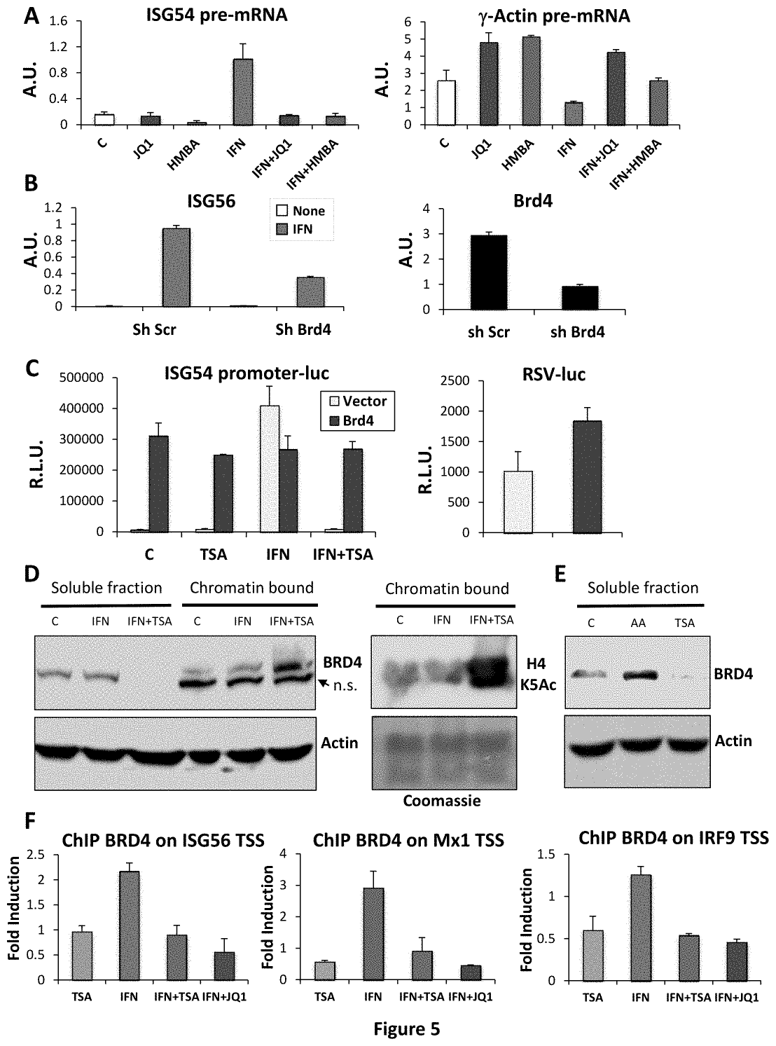

It has been shown that BRD4 coordinates the recruitment of P-TEFb to regulate transcription of target genes, including ISGs. BRD4 recruitment is believed to be triggered by increased histone acetylation at or near transcription start sites. To examine if perturbing the nuclear acetylation state with a potent HDAC inhibitor could impair proper recruitment of BRD4 to ISG promoters, we tested the effect of BRD4 inhibition on ISG expression. In order to focus on transcriptional events, we again monitored induction of ISG unspliced pre-mRNA. JQ-1, a BET protein domain selective inhibitor that impairs BRD4 recruitment, blocked induction of ISG54 nascent transcripts in IFN-treated cells (FIG. 5A, left panel), without affecting the nuclear abundance of constitutively expressed γ-actin nascent transcripts (right panel). Similarly, hexamethylene bisacetamide (HMBA), another inhibitor of BET bromo-domain proteins, blocked ISG transcription without affecting γ-actin expression (FIG. 5A). Because these agents inhibit multiple BET proteins, we examined the specific role of BRD4 by RNA interference. BRD4 was depleted by using a lentiviral-transduced hairpin, reducing BRD4 mRNA levels by greater than 80% (FIG. 5B). Depletion of BRD4 significantly impaired ISG induction in response to IFN (FIG. 5B). Conversely, we tested the effect of BRD4 overexpression on ISG transcription by monitoring the activity of ISG54-luciferase. Overexpression of BRD4 significantly increased ISG54 promoter activity in the absence of IFN treatment (FIG. 5C, left panel), but showed only a modest stimulatory effect on the constitutive promoter from Rous Sarcoma Virus (RSV) (FIG. 5C, right panel). Surprisingly, Brd4-driven ISG54 promoter activity was largely resistant to HDAC inhibition. No significant differences in Brd4-driven expression were observed in samples with or without TSA and IFN. In contrast, IFN-driven ISG54 promoter activity was completely suppressed by TSA. This result suggests that high amounts of available BRD4 protein led to its facilitated recruitment to ISG promoters, allowing unregulated transcription at least from transiently-transfected DNA templates.

BRD4 is recruited to active promoters along with polymerase, and it associates with elongating polymerase to affect transcriptional elongation. To determine if TSA treatment can amplify bromodomain binding sites in chromatin which could lead to trapping of BRD4 by acetylated chromatin and thereby limiting its accessibility to ISG promoters, we examined global BRD4 distribution. Cells were treated with IFN in the presence or absence of TSA, and nuclear BRD4 was quantified in soluble versus chromatin-bound fractions by differential salt extraction (FIG. 5D). Most BRD4 was detected in the soluble nucleoplasm fraction in untreated or IFN-treated cells (FIG. 5D, compare lanes 1 and 2 with 5 and 6). In contrast, soluble BRD4 was undetectable in nuclei of TSA-treated cells (lane 3). Instead, BRD4 was exclusively found in the chromatin-bound fraction (lane 6). Treatment with TSA led to a large increase in histone acetylation (middle panel). In particular, we detected a large increase in acetylated H4K5, a selective binding site for BRD protein bromodomains.

To test if continued histone acetyltransferase (HAT) activity is needed to drive chromatin acetylation, which is opposed by constitutive HDAC activity, we analyzed BRD4 solubility in nuclei from cells treated with the general HAT inhibitor, anacardic acid. Soluble BRD4 was enriched by this treatment (FIG. 5E, lane 2), opposite to its depletion by TSA (lane 3). These results indicate that blocking ongoing HDAC activity allows BRD4 to accumulate on acetylated nucleosomes, removing it from a more soluble available pool, while blocking constitutive HAT activity does the opposite, consistent with the notion that the steady-state levels of available BRD4 are maintained by the balance between constitutive HAT and HDAC activities.

To directly assess the effect of IFN and HDAC inhibition on BRD4 recruitment, we assayed the presence of BRD4 at ISG promoters by ChIP. BRD4 was recruited to three typical ISG promoters after 1 h of IFN stimulation. Importantly, this recruitment was abrogated by TSA, and, by JQ-1 (FIG. 5F). We also performed global ChIP-Seq for BRD4 recruitment. 366 sites of IFN-dependent BRD4 recruitment were detected, all by 4 of which were abrogated in response to combined IFN and TSA treatment (data submitted to GEO). Enrichr pathway analysis (Kuleshov et al., 2016, Nucleic Acids Res. 44:W90-97) showed significant enrichment of genes regulated in virus-infected cells (p<0.0015) and genes regulated by IFN (p<0.00015).

### Example 7

Combined HDAC and BRD4 Inhibition as a Therapy for Type I Interferonopathies.

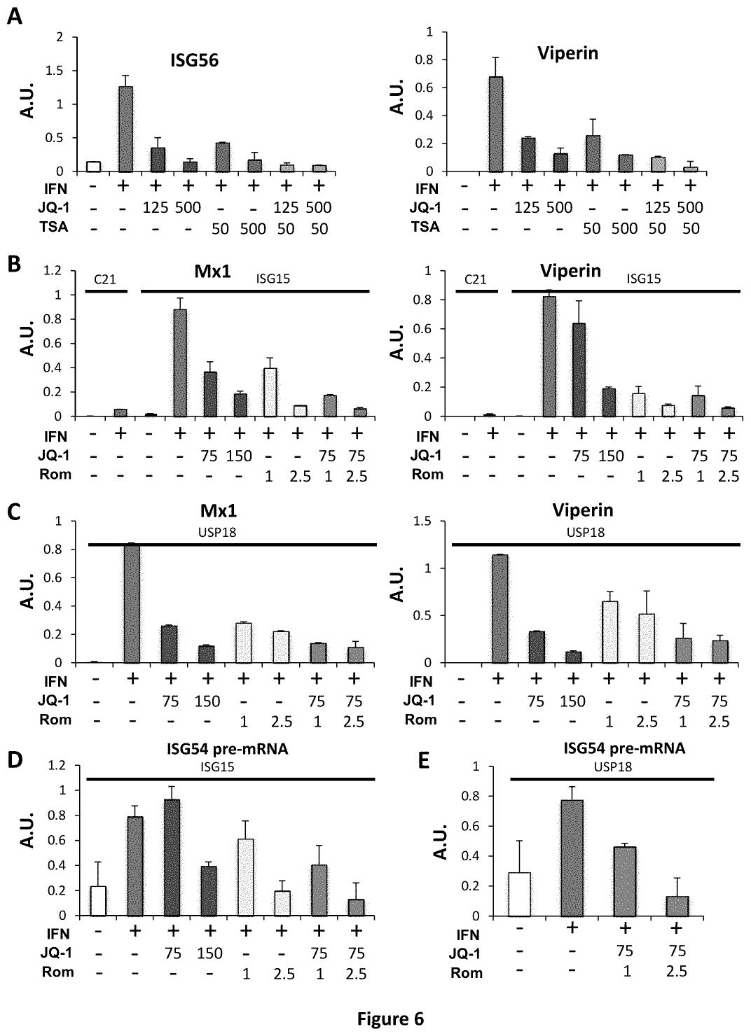

Type I interferonopathy refers to a group of monogenic autoinflammatory diseases in which a constitutive upregulation of type I IFN production or signaling is associated with pathogenesis. It includes diseases such as Aicardi-Goutiéres syndrome (AGS), spondyloenchondromatosis (SPENCD) or familial Chilbain lupus (FCL). Similarly, a common feature of most systemic lupus erythematosus (SLE) patients is elevated serum levels of type I IFN as well as a broad ISG signature. Current therapeutic strategies focus on direct blockade of IFN, its receptors or the downstream signaling pathway (Kirou and Gkrouzman, 2013, Clin. Immunol. 148:303-312). However, directly targeting ISG expression in these patients could also prove effective. Given our results on ISG inhibition by HDAC and BRD4 inhibitors, we determined if combined inhibitor treatment could efficiently thwart ISG expression and could be useful for combinatorial treatment approach to obtain efficacy at reduced concentrations. As a proof of principle, we treated IFN stimulated HEK 293T cells with TSA, JQ-1 or a combination of both drugs. As predicted, a combination of JQ-1 and TSA even at low concentrations (as low as 125 nM for JQ-1 and 50 nM for TSA) almost fully inhibited expression of ISG (FIG. 6A). We extended this study to hTERT immortalized fibroblasts from a patient harboring symptoms of type I interferonopathy, in particular marked intracranial calcification. This patient presented with a single homozygous mutation in the negative regulatory ISG15 gene, resulting in complete loss of the protein. ISG15 is an interferon inducible ubiquitin like protein that can be conjugated to many intracellular proteins by ISGylation. Moreover, ISG15 promotes stability and function of USP18, an isopeptidase and potent negative regulator of IFNα receptor signaling. ISG15 deficient fibroblasts failed to downregulate IFN signaling and therefore exhibited heightened expression of a subset of ISGs, including Mx1 and Viperin (Zhang et al., 2015, Nature 517:89-93). As shown in FIG. 5B, high levels of Mx 1 and Viperin mRNA present in ISG15 deficient fibroblasts were normalized to levels comparable to those observed in control fibroblasts isolated from a healthy individual, after combined treatment with romidepsin and JQ-1. Likewise, hTERT fibroblasts derived from a patient deficient for USP18 were also sensitive to co-treatment with JQ-1 and romidepsin (FIG. 6C), indicating the usefulness of this approach for a range of patients with SLE or type I interferonopathies. It is important to note that the effective drug doses used in these assays were low, making the combination particularly potent. To ascertain that the target of this drug combination was primary transcription, we scored the abundance of ISG54 primary transcripts. This experiment confirmed that ISG54 pre-mRNA was effectively inhibited by a combination of JQ-1 and romidepsin in both ISG15 and USP18 deficient patient fibroblasts (FIGS. 6D and E). Of note, combined HDAC/BRD4 inhibition of IFN-stimulated cells reduced ISG expression in mutant cells to levels similar to wild type.

### Example 8

We have established immortalized B lymphocyte cell lines from healthy donors and from patients with different autoimmune manifestations (Table 1). These cell lines have been evaluated for (1) IFN responsiveness and (2) sensitivity to pathway inhibitors. The data demonstrate robust IFN responses in all cell lines, indicating the measurements of inhibitor efficacy can be made

TABLE 1

| Barcode | Specimen Type | Age | DX |
|---|---|---|---|
| S-180717-00116 | EBV-B cell | 31 | UAS Minor |
| S-181101-00255 | EBV-B cell | 37 | UAS |
| S-181115-00007 | EBV-B cell | 30 | SLE Renal |
| S-181204-00190 | EBV-B cell | 32 | SLE/SS |
| 182311 | EBV-B cell | 33 | Control |
| 182345 | EBV-B cell | 34 | Control |

To obtain data for this Example, B lymphocytes from patient-derived peripheral blood leukocytes were immortalized by infection with Epstein-Barr virus and expanded in tissue culture. Individual cultures were stimulated with IFN in the presence and absence of pathway inhibitors, as discussed above, and RNA was extracted and analyzed for ISG induction by quantitative PCR.

In one approach, peripheral blood leukocytes from symptomatic and asymptomatic patients and from healthy donors are stimulated in vitro with IFN in the presence and absence of pathway inhibitors, as described above. Analysis of responsiveness is performed by multiparameter flow cytometry coupled with mass spectroscopy (CyTOF). IFN-responsive cells are detected by induced expression of the cell surface marker CD169 and their expression profile quantified by staining with antibodies against intracellular and cell-surface markers of IFN responsiveness (Tables 2 and 3). Individuals who exhibit IFN-responsive cells can be treated with the compositions and methods described herein.

TABLE 2
Immunophenotyping

| | |
|---|---|
| Y89Di | CD45 |
| In113Di | CD57 |
| In115Di | CD11c |
| Pr141Di | IgD |
| Nd142Di | CD19 |
| Ce142Di | CD45RA |
| Nd144Di | CD141 |
| Nd145Di | CD4 |
| Nd146Di | CD8 |
| Sm147Di | CD20 |
| Nd148Di | CD16 |
| Sm149Di | CD127 |
| Nd150Di | CD1c |
| Eu151Di | CD123 |
| Sm152Di | CD66b |
| Eu153Di | PD1 |
| Sm154Di | CD86 |
| Gd155Di | CD27 |
| Gd156Di | PDL1 |
| Gd158Di | CD33 |
| Tb159Di | CD24 |
| Gd160Di | CD14 |
| Dy161Di | CD56 |
| Dy162Di | CD169 |
| Dy163Di | CXCR5 |
| Dy164Di | CD69 |
| Ho165Di | CCR6 |
| Er166Di | CD25 |
| Er167Di | CCR7 |
| Er168Di | CD3 |
| Tm169Di | CX3CR1 |
| Er170Di | CD38 |
| Yb171Di | CD161 |
| Yb172Di | CD209 |
| Yb173Di | CXCR3 |
| Yb174Di | HLADR |
| Lu175Di | Axl |
| Lu176Di | Lu176Di |
| Yb176Di | CCR4 |
| Ta181Di | Ta181Di |
| Os189Di | Os189Di |
| Ir191Di | DNA |
| Ir193Di | DNA |
| Bi209Di | CD11b |

TABLE 3
Intracellular staining

| | |
|---|---|
| Y89Di | CD45 |
| In115Di | CD11c |
| Nd142Di | CD19 |
| Nd143Di | CD45RA |
| Nd145Di | CD4 |
| Nd146Di | CD8 |
| Sm147Di | pSTAT5 |
| Nd148Di | CD16 |
| Sm149Di | pSTAT6 |
| Nd150Di | CD1c |
| Eu151Di | CD123 |
| Sm152Di | CD66b |
| Eu153Di | pSTAT1 |
| Gd155Di | CD27 |
| Gd156Di | pp38 |
| Gd158Di | pSTAT3 |
| Tb159Di | pMAPKAP2 |
| Gd160Di | CD14 |
| Dy161Di | CD56 |
| Dy163Di | CD18-APC |
| Ho165Di | STAT3 |
| Er168Di | CD3 |
| Tm169Di | STAT1 |
| Er170Di | CD38 |
| Yb171Di | pERK |
| Yb174Di | HLADR |
| Lu175Di | pS6 |
| Ir191Di | DNA |
| Ir193Di | DNA |

### Example 9

The following materials and methods were used to obtain data presented herein.

Cell Culture

HeLa S3, HEK293, HEK293T, 2fTGH, FS2 human diploid fibroblasts, BT474 (a kind gift from Ben Neel, NYU School of Medicine), Sin3A and Sin3B conditionally-deficient immortalized mouse fibroblasts (a kind gift from Gregory David, NYU School of Medicine) and patient derived hTERT immortalized human fibroblast cells (kindly provided by Dusan Bogunovic, Mount Sinai Icahn School of Medicine) were maintained in DMEM supplemented with 10% calf serum and antibiotics. Sin3A and Sin3B conditional cells expressed tamoxifen-inducible Cre recombinase which was activated by incubation of cells for 3d with 100 nM 4-hydroxytamoxifen (Sigma-Aldrich). Transfections of cells were performed by using the calcium phosphate method, and cellular extracts were collected for protein and RNA analysis. Where indicated, cells were also treated with IFN-α2a (Hoffman-La Roche) at 1,000 units/ml, TSA (Reagents Direct, Encinitas, Calif.) at 150 or 500 ng/ml or as indicated, romidepsin (depsipeptide) (a kind gift from Fujisawa Pharmaceuticals) at 50 or 250 nM, flavopiridol (Selleckchem, Houston, Tex.) at 100 or 200 nM, JQ-1(+), as indicated, RGFP233, RGFP966 (Selleckchem) at 5 or 10 μM, geldanamycin (Sigma-Aldrich) at 2004, hexamethylene bisacetamide (HMBA) (Sigma-Aldrich) at 10 mM, or anacardic acid (EMD Millipore) at 200 μM. Unless otherwise indicated, IFN-α treatments were for 6 h, and inhibitors were added 15 min prior to IFN stimulation. Expression constructs for BRD4 and HDAC1 were obtained separately.

In Vitro Transcription Measurements

In vitro transcription run-off assays were performed as follows. HEK293 cells were used as a source of nuclear extract, supplemented with recombinant ISGF3 produced by transfection. Recombinant ISGF3 was produced by transfecting HEK293 cells with expression plasmids for STAT1, STAT2, IRF9, and JAK1. Transcription extracts were programmed with HindIII-digested ISG54 p107, containing promoter sequences from −547 to +283 of the human ISG54 gene or PvuII-digested G6TI-CAT control DNA template containing 6 SP1 sites derived from the SV40 early promoter.

Run-on nuclear transcription experiments were conducted as follows. 10 million HeLa S3 cells were used per point. Nuclei were isolated from cells in RSBG40 lysis buffer (10 mM Tris, pH 7.4, 10 mM NaCl, 3 mM MgCl₂, 10% glycerol, and 0.25% NP40, 0.5 mM DTT, 0.5 mM PMSF, 1× protease inhibitor cocktail). Polymerase elongation was released in the presence of radiolabeled UTP, with or without addition 150 ng/ml TSA. Radiolabeled nuclear RNA was isolated by TRIzol reagent (Invitrogen). 6 μg of plasmid DNA was spotted onto nitrocellulose for slotblot hybridization and radioactive signals were quantified by phosphorimaging (Bio-Rad). DNA probes used were the following: Vector (pGEM1, Promega), γ-actin cDNA, GAPDH, ISG15 TaqI fragment from exon 2, ISG56, IRF9, GBP, 6-16 and 9-27.

Protein Assays

Nuclear extracts were prepared and analyzed by immunoblotting as follows. Cells were lysed in RSBG40, and soluble nuclear proteins were extracted from pelleted nuclei in RSB supplemented with 150 mM NaCl, and chromatin-bound proteins were subsequently extracted in 400 mM NaCl buffer. Alternatively, whole cell lysates were used. Antibodies used were anti-IRF9, anti-STAT1, anti-STAT2, anti-phospho-STAT1 and anti-phospho-STAT2 (Invitrogen), anti-α-tubulin (T9026, Sigma), anti-HDAC1 (clone 2E10, #05-614, MilliporeSigma), anti-HDAC2 (clone 3F3, #05-814, MilliporeSigma), anti-E2F4 (A-20, sc-1082, Santa Cruz Biotechnology), anti-Sin3B (AK-12, Santa Cruz), anti-NELF-E (H140, Santa Cruz), anti-BRD4 (A-301-985A100, Bethyl Laboratories), anti-Actin (Clone C4, MAB1501, MilliporeSigma). Luciferase assays were performed using standard methods.

Real-Time RT-PCR

Cytoplasmic and nuclear RNA was isolated and converted to cDNA. Relative abundance of specific mRNA sequences was determined by real-time fluorescent PCR, by using SYBR Green (Molecular Probes), with comparison to a standard curve generated by serial dilution of a cDNA sample containing abundant target sequences and normalization to the expression of GAPDH. All PCR reactions were performed in triplicate, PCR efficiencies were greater than 85%, linearity of standard curves was determined by least-squares linear regression, and standard errors were typically <10% of mean values. Sequences of primers used are available on request.

ChIP-qPCR and ChIP-seq

Chromatin immunoprecipitation was performed as follows. Cells from two 15-cm plates (˜40 million) were treated with 1% formaldehyde for 30 min at 4° C. and fixation was quenched by addition of 125 mM glycine. Fixed cells were lysed in 10 ml of cold Buffer A (50 mM Hepes, pH 7.4, 1 mM EDTA, 0.5 mM EGTA, 140 mM NaCl, 10% glycerol, 0.5% NP-40, and 0.25% Triton-X-100), and nuclei were collected by centrifugation and washed with 10 ml of cold Buffer B (10 mM Tris-HCl, pH 8.0, 1 mM EDTA, 0.5 mM EGTA, and 200 mM NaCl). Nuclei were extracted in 2 ml of cold Buffer C (10 mM Tris-HCl, pH 8.0, 1 mM EDTA, 0.5 mM EGTA, 140 mM NaCl, 5% glycerol, 0.1% sodium deoxycholate, 0.1% SDS, and 1% Triton-X-100). Genomic DNA was sheared by sonication, either by using Sonic Dismembrator Model 500 (Fisher Scientific) in a dry ice ethanol bath, or by using BiorupterPlus (Diagenode) in a 4° C. water bath, to achieve chromatin fragments between 100-500 bp. Sheared chromatin from 4-10 million cells was used for each immunoprecipitation. Immunoprecipitates were collected on protein A Sepharose or protein A magnetic beads and digested with proteinase K in 100 μl of Buffer D (10 mM Tris-HCl, pH 8.0, 100 mM NaCl, 0.5% SDS, 25 mM EDTA, 100 μg/ml proteinase K) at 55° C. for 3 hours. Crosslinks were reversed by incubating the digested chromatin at 65° C. overnight. Immunoprecipitated DNA sequences were recovered by alcohol precipitation and quantified by real-time PCR compared with input genomic DNA.

ChIP-Seq libraries were made using the KAPA Hyper Prep kit (Roche, KK8504) using 25 ng of DNA per sample, according to the manufacturer's protocol for end repair and A-tailing. For adapter ligation, 8-nt multiplexing IDT (Integrated DNA Technologies) adapters (5 μM) were used. 0.8× post-ligation cleanup was performed with AMPure XP beads (Beckman Coulter) on adapter-ligated libraries. Libraries were size selected prior to PCR amplification with a 0.6× right-side selection and 1.2× re-bind and cleanup. 6 PCR cycles were used for library amplification. Libraries were sequenced on an Illumina HiSeq 4000 instrument, using a single read 50 protocol; 8 samples were pooled in one lane of a high output single read flow cell. Sequencing results were demultiplexed and converted to FASTQ format using Illumina Bc12Fastq software.

ChIP-Seq data analysis was carried out using the HiC-Bench pipeline (Lazaris et al., 2017, BMC Genomics 18:22). GenomicTools (Tsirigos et al., 2012, Bioinformatics 28:282-283), SAMtools (Li et al., 2009, Bioinformatics 25:2078-2079), DeepTools (Ramirez et al., 2016, Nucleic Acids Res. 44:W160-165), BEDTools (Quinlan and Hall, 2010, Bioinformatics 26:841-842), BigBed Tools (Kent et al., 2010, Bioinformatics 26:2204-2207), Picard Tools (https://broadinstitute.github.io/picard/), and R (https://www.R-project.org/) were used to analyze the sequenced reads and to check the quality of alignment. Bowtie2 (Langmead, 2010, Bioinformatics Chapter 11:Unit 11 17) was used to align the raw reads to the hg19 reference genome. After alignment, MACS2 peak caller (Feng et al., 2012, Nat Protoc 7:1728-1740) was used to identify enriched peaks and these peaks were visualized in the UCSC Genome Browser (Kent et al., 2002, Bioinformatics 26:2204-2207.). DiffBind (Ross-Innes et al., 2012, Nature 481:389-393) package was used to determine the differentially bound peaks (p<0.001). The Intervene tool (Khan and Mathelier, 2017, BMC Bioinformatics 18:287) was used to compare peak overlaps between treatment groups.

Antibodies that were used for ChIP included: 2 μg of anti-RNA Pol-II Ab (N-20, sc-899, Santa Cruz Biotechnology), 5 μl of anti-RNA Pol-II H14 (serine-5) (MMS-134R, Covance), 2 μg of anti H3K4(Me3) (ab85580-25, Abcam), 2 μg of anti-CDK9 (C20, sc-484, Santa Cruz Biotechnology), 5 μg anti-BRD4 (A-301-985A100, Bethyl Laboratories).

MNase-PCR Mucleosome Mapping

FS2 cells were starved for 72 h in DMEM supplemented with 0.02% fetal bovine serum before treatment with IFN-α2a (1,000 units/ml) with or without TSA (1 μg/ml). Cells were fixed with 1% formaldehyde for 30 min at 4° C., lysed in 10 mM Tris-HCl, pH 7.5, 10 mM CaCl₂), 3 mM MgCl₂, 0.4% NP-40, 10 mM sodium butyrate, 0.1 μM bezamidine, 0.5 mM PMSF, 1× protease inhibitor cocktail, and nuclei were collected and resuspended in the same buffer at a final genomic DNA concentration of 1.25 μg/μ1. Nuclei were digested with 4 U/ml micrococcal nuclease for 10-15 minutes at 37° C., and mononucleosomal DNA was purified by agarose electrophoresis. Nuclease protection of specific DNA segments was determined by PCR and normalized to a region from the ε-globin promoter protected by the N1 nucleosome.

RNAi and shRNA Knockdowns

HEK293 cells were transfected twice sequentially with 20 nM siRNA oligonucleotides by using the calcium phosphate precipitation method, and treated with IFN-α and TSA for 6 h. The siRNA oligonucleotides targeted HDAC1 and 2 (Dharmacon SMARTpool® M-003494, L-003495), or E2F4, a kind gift from Brian Dynlacht (NYU School of Medicine). shRNA against NELF-E (clone NM_002904.4-119551c1) and BRD4 (clone NM 058243.1-170751c1) were Mission® shRNA from Sigma-Aldrich and shRNA against Spt5 was a obtained separately.

Discussion of the Foregoing.

The data described in disclosure demonstrate that IFN-stimulated gene transcription depends on HDAC activity to facilitate the transition from committed initiation to processive elongation through, at least in part, the targeted recruitment of P-TEFb to ISG promoter-proximal regions mediated by the BRD4 protein.

We found that HDAC activity was not required to reorganize promoter chromatin in response to IFN, allow chromatin binding of the activated ISGF3 transcription factor complex, recruit RNAPII to ISG promoters and promoter-proximal regions, or to allow transcriptional initiation, as judged by Ser 5 phosphorylation of RNAPII. However, movement of RNAPII for transcriptional elongation and its accumulation in a Ser 2-phosphorylated form was impaired in the absence of HDAC activity, indicative of failed elongation. These findings indicate that HDAC activity is required for the transition to processive elongation, possibly to overcome promoter-proximal pausing. We found evidence for significant promoter-proximal pausing at ISG promoters by the large accumulation of RNAPII at promoter-proximal sites relative to gene bodies (FIG. 3), a common observation for paused genes. However, unlike other instances of promoter pausing, e.g., poised promoters regulated through a pause-release mechanism, ISG promoters only displayed signs of polymerase pausing following IFN stimulation. Thus, these promoters appear to combine two distinct mechanisms of transcriptional control, inducible recruitment of polymerase and promoter-proximal pausing.

Aberrant ISG regulation results in interferonopathies and other autoimmune diseases. There is strong evidence that an ISG expression signature is at least in part causative in such diseases, rather than being simply correlative. Therefore, inhibition of enhanced ISG expression has become an attractive anti-inflammatory therapeutic target, with the caveat that ISG inhibition could increase the risk for viral infection (Kalunian, 2016, Lupus 25:1097-1101). The present disclosure provides that an alternative to abrogating the ISG pathway in autoimmunity is to reduce aberrant constitutive signaling back to physiologic levels. Results presented herein indicate that combined low-dose HDAC and BRD inhibitors, drugs available for clinical use in humans and well tolerated by patients, can provide a viable approach to reducing inflammation without abrogating beneficial IFN signaling.

While the present invention has been described through illustrative embodiments, routine modification will be apparent to those skilled in the art and such modifications are intended to be within the scope of this disclosure.

What is claimed is:

1. A method of treating an autoimmune disease comprising administering to an individual in need of treatment one or more compositions comprising one or more inhibitors of histone deacetylatse (HDAC) and one or more inhibitors of BRD4.

2. The method of claim 1, wherein the HDAC inhibitor and/or the BRD4 inhibitor are administered at a sub-therapeutic dose.

3. The method of claim 2, wherein the sub-therapeutic dose is less than 1 μM.

4. The method of claim 1, wherein the HDAC inhibitor is Romidepsin.

5. The method of claim 1, wherein the BRD4 inhibitor is ARV-825, JQ-1 or Silmitasertib.

6. The method of claim 1, wherein the BRD4 inhibitor and the HDAC inhibitor are administered separately.

7. The method of claim 1, wherein the signaling pathway inhibitor is a specific inhibitor of one or more members of the JAK kinase family.

8. The method of claim 1, wherein the autoimmune disease is systemic lupus erythematosus (SLE) or rheumatoid arthritis (RA).

9. A method of treating an autoimmune disease comprising administering to an individual in need of treatment one or more compositions comprising one or more of the following:
i) an inhibitor of histone deacetylation (HDAC)
ii) an inhibitor of binding of BRD4 to chromatin,
iii) an inhibitor of activation of BRD4 by phosphorylation, and/or
iv) an inhibitor of signaling pathways leading to induction or increased expression of Type I interferon stimulated genes (ISG).

10. The method of claim 9, wherein the inhibitor of HDAC is Romidepsin.

11. The method of claim 9, wherein the inhibitor of BRD4 binding to chromatin or BRDE4 activation is ARV-825, JQ-1 or Silmitasertib.

12. The method of claim 9, wherein the BRD4 inhibitor and the HDAC inhibitor are administered separately.

13. The method of claim 11, wherein the inhibitor of the signaling pathway is a specific inhibitor of one or more members of the JAK kinase family.

14. The method of claim 9, wherein the inhibitors are administered separately or in combination.

15. The method of claim 13, wherein the inhibitors are administered separately or in combination.

16. The method of claim 9, wherein the autoimmune disease is systemic lupus erythematosus (SLE) or rheumatoid arthritis (RA).
